(19) United States
(12) Patent Application Publication
(10) Pub. No.: US 2017/0348089 A1
(43) Pub. Date: Dec. 7, 2017

(54) ADJUSTABLE BREAST IMPLANT WITH INTEGRAL INJECTION PORT

(71) Applicant: TECHNO INVESTMENTS, LLC, Boca Raton, FL (US)

(72) Inventor: HILTON BECKER, BOCA RATON, FL (US)

(21) Appl. No.: 15/344,292

(22) Filed: Nov. 4, 2016

Related U.S. Application Data
(60) Provisional application No. 62/346,980, filed on Jun. 7, 2016.
(60) Provisional application No. 62/355,724, filed on Jun. 28, 2016.

(51) Int. Cl. A61F 2/12

(52) U.S. Cl. CPC A61F 2/12; A61F 2250/0003

(57) ABSTRACT

A breast implant comprising an outer shell that may be filled with saline or a double shell comprising two chambers, wherein the outer shell may be filled with silicone gel and the inner shell may be filled with saline. Alternatively, the single lumen saline chamber may contain several bubble shells disposed within the lumen to baffle the saline, giving the implant a more gel-like feel. The implant may be selectively filled with saline via a self-sealing valve located on the superior edge of the exterior surface of the outer shell. The valve is palpable but small enough so as not to bother the patient. The valve includes an injection port that provides access to the saline lumen so that the size of the shell is adjustable.

### CROSS-REFERENCE TO RELATED APPLICATIONS

The present patent application claims the benefit of U.S. Provisional Application No. 62/346,980, entitled “Adjustable Breast Implant with Integral Injection Port,” which was filed on Jun. 7, 2016 in the name of the inventor herein, Hilton Becker, and which is incorporated herein in full by reference. The present application claims the benefit of U.S. Provisional Application No. 62/355,724, entitled “Integral Injection Port,” which was filed on Jun. 28, 2016 in the name of the inventor herein, and which is also incorporated herein in full by reference.

### FIELD OF THE INVENTION

The present invention generally relates to breast implants, and more specifically, to an adjustable breast implant with an integral injection port.

### BACKGROUND OF THE INVENTION

Breast reconstruction following mastectomy is most commonly performed by placing a tissue expander below the muscle and expanding the overlying remaining tissues. The typical expander is a single lumen saline-filled device which is subsequently replaced with a silicone gel implant. As the expander is placed beneath the muscle it is necessary to have a large injection port in order to locate it beneath the thick tissue. However, the bulkiness of the injection port makes it undesirable for permanent placement. Therefore, it is subsequently replaced with a gel implant.

Now that surgeons are performing skin sparing mastectomies, it is possible to place the expander above the muscle. Therefore, a smaller injection port may be utilized. With a smaller injection port, it is possible to keep the expander in position long term without the injection port being bothersome to the patient.

Double lumen expanders having silicone gel in the outer chamber and saline in the inner chamber have previously been used. For example, Applicant's U.S. Pat. No. 7,081,136 (“'136 Patent”) discloses an adjustable gel-filled mammary prosthesis. That patent discloses that saline is added to the inner chamber via a remote injection port that is removed once filling is completed. The implant seals at a valve once the filling port is removed. Thus, a saline-gel implant becomes the definitive implant without the need to replace the expander with a gel implant. The implant disclosed in the '136 Patent, however, includes the need to place the injection port in a remote pocket. The injection port then needs to be removed at a second procedure. The injection port can become obstructed by kinking of the filling tube. The remote port may also at times become a source of irritation to the patient. It can also erode through the skin and become infected. Therefore, an integral injection port placed in the upper pole of the expander/implant is advantageous.

An improved design is described which overcomes the problems related to a remote port implant. For example, Applicant's U.S. Pat. No. 4,773,908 (“'908 Patent”) discloses an implant having an injection port that is integrally attached to the implant without the need to place it remotely, thus eliminating the associated complications.

Currently available integral tissue expanders are not suitable for pre-pectoral placement and are not designed to remain in place as the definitive implant. The present invention, however, facilitates placement of the implant above the muscle.

The device disclosed herein consists of either a single lumen or double lumen implant containing an integral injection port in the upper pole. The single lumen saline implant may contain multiple bubble shells within the inner chamber in order to buffer the saline. The shells may be spherical, concave, or flat and stacked upon each other in order to create a sponge like structure that baffles the saline resulting in a more gel-like feel to the implant. The single lumen implant may be filled with saline while the double lumen implant may contain silicone gel in the outer lumen and saline in the inner lumen. The shells may be attached to each other by means of an injection port. It is thus possible to inject saline into the inner lumen by injecting into the injection port.

The device may be placed beneath the skin flaps at the completion of the mastectomy. The implant may be placed under-filled, or even empty, if the circulation to the overlying skin flaps appears compromised. Once the circulation has improved, saline is added to the implant via the self-sealing injection port. In order to facilitate palpability of the injection port, the injection port may be placed at the apex of the device which is preferable when the implant is placed above the muscle.

A tab may be attached to the upper pole of the implant below the injection port. A suture may be placed through the tab and brought out through the skin. When saline is injected into the injection port, tension may be placed on the tab, thus securing the port and facilitating location of the port. At the end of the filling procedure, the external suture may be removed. Another option is to detachably adhere the injection port to the implant. It may then removed from the implant which seals by means of a valve as described in the '136 Patent. A small incision is made over the injection port for removal.

### SUMMARY

In accordance with one embodiment of the present invention, an adjustable breast implant is disclosed. The adjustable breast implant comprises: an outer shell; at least one inner lumen within the outer shell, wherein the at least one inner lumen is configured to receive a fluid to adjust a volume of the adjustable breast implant; and an injection port disposed on the outer shell and positioned proximate an upper pole of the adjustable breast implant.

In accordance with another embodiment of the present invention, an adjustable breast implant is disclosed. The adjustable breast implant comprises: an outer shell; at least one inner lumen within the outer shell, wherein the at least one inner lumen is configured to receive a fluid to adjust a volume of the adjustable breast implant; an injection port disposed on the outer shell and positioned proximate an upper pole of the adjustable breast implant, wherein the injection port comprises: a fill reservoir defined by the outer shell; and a fluid tube connected to the fill reservoir; wherein the fill reservoir is in fluid communication with the at least one inner lumen via the fluid tube; a suture tab disposed proximate to the injection port at the upper pole of the adjustable breast implant, wherein the suture tab is absorbable.

In accordance with another embodiment of the present invention, an adjustable breast implant is disclosed. The adjustable breast implant comprises: an outer shell; at least one inner lumen within the outer shell, wherein the at least one inner lumen is configured to receive a fluid to adjust a volume of the adjustable breast implant; an injection port disposed on the outer shell and positioned proximate an upper pole of the adjustable breast implant, wherein the injection port comprises: a fill reservoir defined by the outer shell; and a fluid tube connected to the fill reservoir; an absorbable outer shell barrier; wherein the fill reservoir is in fluid communication with the at least one inner lumen via the fluid tube; an absorbable suture tab disposed proximate to the injection port at the upper pole of the adjustable breast implant; a plug positioned within the outer shell; a hollow flow tube having one end coupled to the plug and having another end coupled to the injection port, wherein the fluid flows from the injection port through the hollow tube into the adjustable breast implant; and an aperture disposed on the flow tube to provide a flow path of the fluid into the adjustable breast implant; wherein the injection port is removable from the plug via the flow tube whereby exerting a tensile force onto the injection port separates the injection port from the plug, and the plug seats against the outer shell in response to removal of the injection port due to the tensile force.

Disclosed is an adjustable breast implant comprising: an outer shell; an inner lumen bounded by the outer shell to receive a fluid to adjust a volume of the adjustable breast implant; an injection port disposed on the outer shell and arranged at the upper pole of the adjustable breast implant; and a suture tab disposed proximate to the injection port at the upper pole of the adjustable breast implant.

Further disclosed is an adjustable breast implant comprising: an outer shell; an inner lumen bounded by the outer shell to receive a fluid to adjust a volume of the adjustable breast implant; a plurality of bubble shells disposed in the inner lumen, the bubble shells being spaced apart from the outer shell and disposed in the inner lumen a nested arrangement with respect to one another such that the bubble shells are spaced apart from each other by a plurality of second inner lumens, each second inner lumen being interposed between neighboring bubble shells, each of the bubble shells comprising a three-dimensional sinusoidal wall, and the bubble shells being size-adjustable, based on an amount of fluid disposed therein; an injection port disposed on the outer shell and arranged at an upper pole of the adjustable breast implant, the injection port to receive the fluid and to communicate the fluid to the inner lumen; and a suture tab disposed proximate to the injection port at the upper pole of the adjustable breast implant, wherein the injection port provides fluid access to inflate and adjust a size of the inner lumen and second inner lumens.

Further disclosed is an adjustable breast implant comprising: an outer shell containing an inner shell. The first inner lumen is filled with silicone gel 25 to 80% of the total volume of the outer shell. The inner shell is attached to the outer shell at the entry point of the filling port. The volume of the inner shell is between 25 and 75% of the volume of the outer shell.

### BRIEF DESCRIPTION OF THE DRAWINGS

The present application is further detailed with respect to the following drawings. These figures are not intended to limit the scope of the present application, but rather, illustrate certain attributes thereof.

FIG. 1 is a front cross-sectional view of an adjustable breast implant that includes an outer shell in accordance with one embodiment of the present invention;

FIG. 2 shows a cross-section of the adjustable breast implant of FIG. 1;

FIG. 3 is a front cross-sectional view of an adjustable breast implant that includes an inner shell disposed in an outer shell in accordance with one embodiment of the present invention;

FIG. 4 shows a cross-section of the adjustable breast implant of FIG. 3;

FIG. 5 is a front cross-sectional view of an adjustable breast implant that includes an anatomical shape and a plurality of nested bubble shells disposed in an outer shell in accordance with one embodiment of the present invention;

FIG. 6 shows a side cross-sectional view of the adjustable breast implant of in FIG. 5 shown with the implant under-filled;

FIG. 7 shows a side cross-sectional view of the adjustable breast implant of in FIG. 5 shown with the implant filled with saline;

FIG. 8 is a side cross-sectional view of an adjustable breast implant that includes an anatomical shape and a plurality of bubble sheets disposed in an outer shell in accordance with one embodiment of the present invention;

FIG. 9 shows a side cross-sectional view of the adjustable breast implant of FIG. 8 shown with saline disposed in an outer shell;

FIG. 10 shows a side cross-sectional view of a removable integral filling port disposed in an outer shell of an adjustable breast implant;

FIG. 11 is a side cross-sectional view of the integral filling port of FIG. 10 shown with an injection port detached from the implant and wherein the implant is sealed with a plug;

FIG. 12 is a perspective view of a prior art expander;

FIG. 13 is a perspective view of an adjustable breast implant with a resorbable barrier and a micro-injection port in accordance with one embodiment of the present invention;

FIG. 14 is a perspective view of the adjustable breast implant of FIG. 13 shown with the outer shell barrier resorbed;

FIG. 15A is a side view of a prior art expander with a fixed injection port;

FIG. 15B is a side view of a prior art expander with an unattached injection port;

FIG. 16 is a side view of an adjustable breast implant with a fixed retractable injection port in accordance with one embodiment of the present invention;

FIG. 17A is a side view of an adjustable breast implant with a fixed retractable injection port and with absorbable suture tabs placed at the edges of the adjustable breast implant in accordance with one embodiment of the present invention; and

FIG. 17B is a side view of the adjustable breast implant of FIG. 17A shown with the injection port retracted to a desired length.

### DETAILED DESCRIPTION OF THE INVENTION

The description set forth below in connection with the appended drawings is intended as a description of presently preferred embodiments of the disclosure and is not intended to represent the only forms in which the present disclosure may be constructed and/or utilized. The description sets forth the functions and the sequence of steps for constructing and operating the disclosure in connection with the illustrated embodiments. It is to be understood, however, that the same or equivalent functions and sequences may be accomplished by different embodiments that are also intended to be encompassed within the spirit and scope of this disclosure.

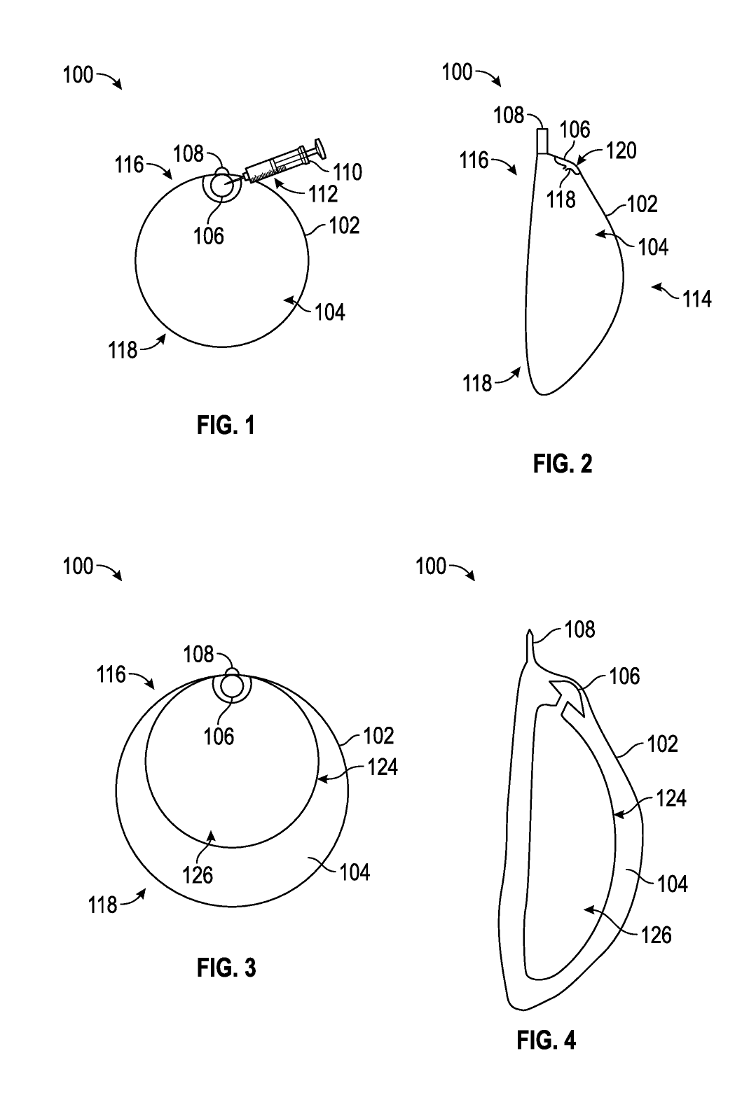

FIGS. 1-17B together, disclose an adjustable breast implant 100 having a superior surface 114, an upper pole 116, and a lower pole 118, wherein the adjustable breast implant 100 has an integral injection port 106 disposed on the outer shell 102 proximate the upper pole 116. The integral injection port 106 comprises a fill reservoir 120 that is defined by the outer shell 102 and a fluid tube 122 that is connected to the fill reservoir 120 and leads to and is in fluid communication with the inner lumen 104. The injection port 106 may also have a puncture resistant back or shell barrier 140 that prevents a needle from damaging the adjustable breast implant 100. Referring to FIGS. 1-2, the adjustable breast implant 100 can include one inner lumen 104, which is defined by an outer shell 102. The inner lumen 104 may be filled with saline 112 or another suitable fluid in order to adjust the volume of the adjustable breast implant 100. The inner lumen 104 may also be filled with any suitable gas such as air, nitrogen, and carbon dioxide. Alternatively, as shown in FIGS. 3-4, the adjustable breast implant 100 can include a first inner lumen 104 that is defined by an outer shell 102 and can also include a second inner lumen 126 that is defined by an inner shell 124. In the double lumen adjustable breast implant 100 shown in FIGS. 3-4, the first inner lumen 104 may contain silicone gel and the second inner lumen 126 may contain saline 112. The inner shell 124 prevents the two fluids from mixing by blocking fluid communication between the first inner lumen 104 and the second inner lumen 126. The outer shell 102 and the inner shell 124 may be attached to each other by the injection port 106. Therefore, saline 112 may be injected into the second inner lumen 126 by injecting the saline 112 with a syringe 110 into the injection port 106. The first inner lumen 104 may be filled with silicone gel 25 to 80% of the total volume of the outer shell 102. The volume of the inner shell 124 may be between 25 and 75% of the volume of the outer shell 102.

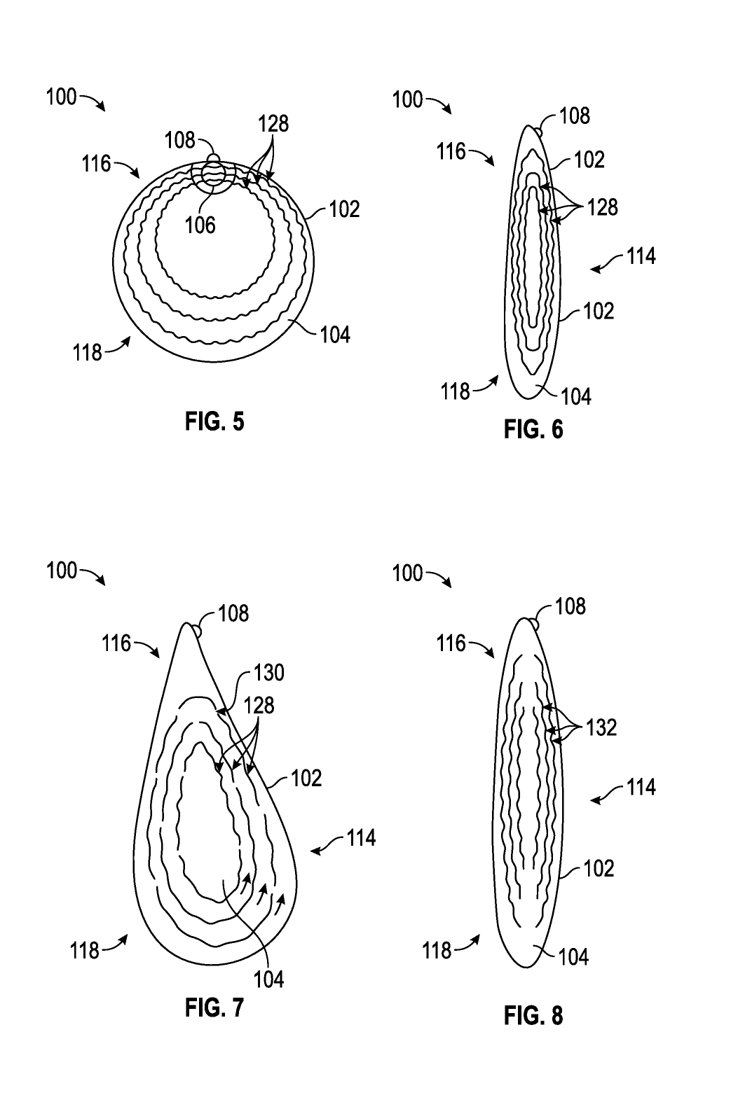

Referring to FIGS. 5-7, the adjustable breast implant 100 may include a plurality of bubble shells 128 within a first inner lumen 104. The bubble shells 128 may be spaced apart from the outer shell 102 and positioned within the first inner lumen 104 in a nested arrangement with respect to one another such that the bubble shells 128 are spaced apart from each other by a plurality of second inner lumens 126, each second inner lumen 126 being interposed between neighboring bubble shells 128. Each bubble shell 128 comprises a three-dimensional sinusoidal shaped wall and each bubble shell 128 is size-adjustable so that it expands when the adjustable breast implant 100 is filled with fluid. The injection port 106 provides fluid access to inflate and adjust a size of the first inner lumen 104 and the second inner lumens 126. The bubble shells 128 help to baffle motion of the saline 112 within the first inner lumen 104 of the adjustable breast implant 100 such that movement of the fluid within the outer shell 102 and among the bubble shells 128 is slowed as compared to an absence of the bubble shells 128 or an absence of the sinusoidal wall. The bubble shells 128 can be spherical, concave, flat, and the like and can be stacked upon each other in order to create a sponge-like structure that baffles the saline 112 to provide the adjustable breast implant 100 with a gel-like feel. The bubble shells 128 may also have openings 130 to allow the saline 112 to move freely in and out of them.

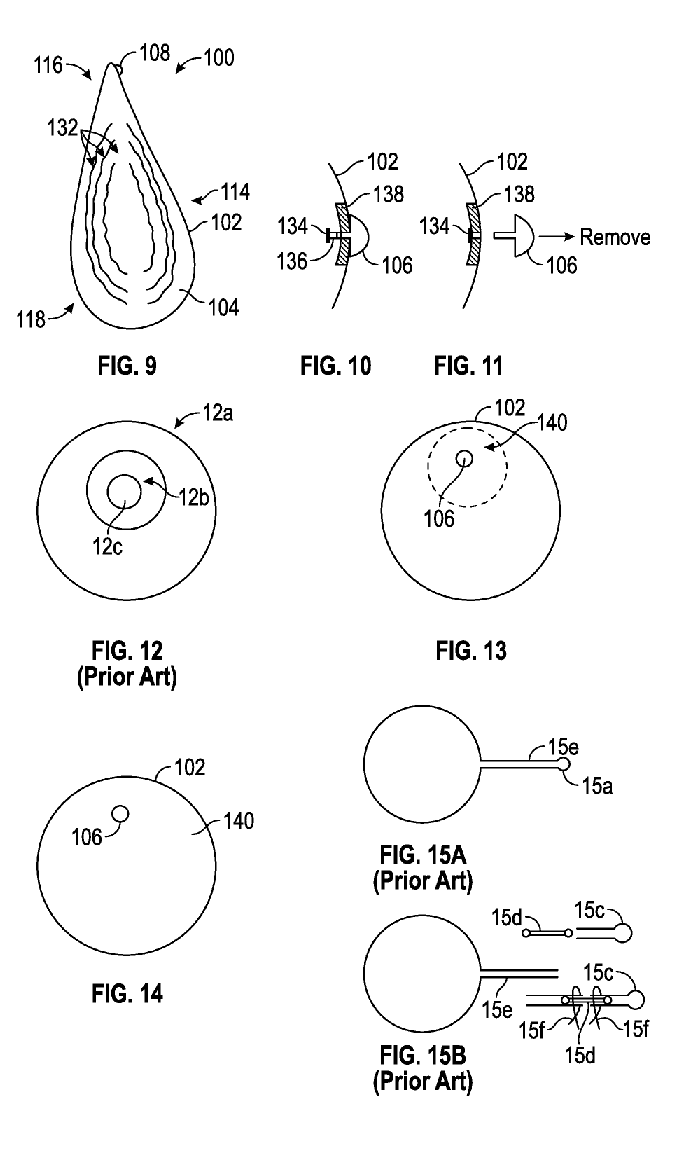

Referring to FIGS. 8-9, the adjustable breast implant 100 may include a plurality of bubble sheets 132. The bubble sheets 132 may be corrugated or have sinusoidal surfaces substantially similar to the bubble shells 128. These bubble sheets 132 also may be stacked upon each other so that they baffle the saline 112 to provide the adjustable breast implant 100 with a gel-like feel.

Referring to FIGS. 10-11, saline 112 may be added to the inner lumen 104 via the integral injection port 106, which is permanently affixed to the outer shell 102. In some embodiments, the injection port 106 protrudes from the outer shell 102 and may be removed, and a plug 134 may be used to seal the adjustable breast implant 100. The plug 134 may be positioned within the outer shell 102. A flow tube 136 may be interposed between and in physical contact with the plug 134 and the injection port 106. The flow tube 136 may be a hollow tube to allow fluid, such as saline 112, to flow from the injection port 106 into the adjustable breast implant 100. An aperture may also be disposed on the flow tube 136 to provide a flow path of the fluid into the adjustable breast implant 100.

The injection port 106 may be removable from the plug 134 via the flow tube 136 such that exerting a tensile force onto the injection port 106 from the plug 134 separates the injection port 106 from the plug 134 and the plug 134 seats against the inner surface of the outer shell 102 in response to removal of the injection port 106 due to the tensile force. The adjustable breast implant 100 therefore seals at the plug 134 in response to removal of the injection port 106. Thus, the adjustable breast implant 100 is a definitive implant that is permanent in an absence of replacement of the adjustable breast implant 100 with another implant (e.g., a gel implant). The adjustable breast implant 100 may also have a sealing patch 138 that may be disposed on the outer shell 102 to seal the adjustable breast implant 100 and prevent leakage of the fluid (e.g. saline 112) from the adjustable breast implant 100. The sealing patch 138 may sealingly engage the injection port 106 prior to removal of the injection port 106 and sealingly engage the plug 134 in response to removal of the injection port 106.

As shown in the embodiments of FIGS. 1-9, the injection port 106 may be flush with the adjustable breast implant 106 and may be removed at a self-sealing valve disposed in an upper pole 116 of the adjustable breast implant 100. In order to facilitate palpability of the injection port 106, the injection port 106 may be placed at the apex of the upper pole 116 of the adjustable breast implant 100, which is preferable when the adjustable breast implant 100 is placed above the pectoral muscle. The integral injection port 106 placed in the upper pole 116 of the adjustable breast implant 100 is advantageous because the adjustable breast implant 100 can be placed beneath the skin flaps, above the pectoral muscle, at the completion of the mastectomy. The adjustable breast implant 100 may be positioned when it is under-filled, or even empty, if the circulation to the overlying skin flaps is compromised. Once the circulation has improved, saline 112 can be added to the adjustable breast implant 100 via the self-sealing injection port 106.

A suture tab 108 may be disposed at the upper pole 116 of the adjustable breast implant 100 proximate to the injection port 106. A suture may be placed through the suture tab 108 and brought out through the skin. When saline 112 is injected into the injection port 106, tension may be placed in the suture tab 108 to secure the injection port 106 and facilitate location of the injection port 106. At the end of the filling procedure, the external suture can be removed.

In a certain embodiment, the injection port 106 may be detachably adhered to the adjustable breast implant 100. The injection port 106 may be removed from the adjustable breast implant 100, and the adjustable breast implant 100 sealed by a valve such as the plug 134. An incision in the patient can be made over the injection port 106 to remove the injection port 106 from the adjustable breast implant 100. An advantage is that the adjustable breast implant 100 has the injection port 106 that is initially integrally attached to the adjustable breast implant 100 which is then subsequently removed, as opposed to the injection port 106 being remotely attached to the adjustable breast implant 100. This helps to facilitate placement of the adjustable breast implant 100 above the pectoral muscle.

In FIG. 12, a prior art expander is shown. The standard expander has an outer shell 12a, a protective barrier 12b, and a very large injection port 12c. The standard integral injection port expander, which is designed to be placed beneath the pectoral muscle, has a large bulky and palpable injection port 12c as well as a large protective barrier 12b, which is palpable to the patient and therefore not suitable for long term implantation. FIGS. 13-14 show the adjustable breast implant 100 of the present invention. The adjustable breast implant 100 with integral injection port 106 is shown having an outer shell 102, an absorbable outer shell barrier 140, and a very small injection port 106 (i.e. a microinjection port). In FIG. 14, the absorbable outer shell barrier 140 is shown as having been absorbed. An important object of this invention is to have the smallest possible integral injection port 106 that will not be palpable by the patient. As the injection port 106 is small, a protective shell barrier 140 is necessary to prevent accidental needle puncture when filling the adjustable breast implant 100. The invention disclosed herein proposes a small injection port 106 protected by an absorbable protective barrier 140. The barriers 140 can be constructed of a resorbable alloplastic material such as poly lactic acid, polyglactin 910, polydioxanone (PDS®), glycolide/lactide copolymer (POLYSORB®), or other similar absorbable polymer. It is only necessary to access the injection port 106 for a few weeks after surgery so the absorbable barrier 140 resorbs at about 3-6 months after surgery. By that time, the barrier 140 is no longer palpable and the remaining implant 100 is soft and natural feeling.

In FIGS. 15A-15B, prior art expanders are shown. In FIG. 15A, the injection port 15a may be fixed to the filling tube 15e which is attached to the expander. This results in the distance between the injection port 15a and the expander being fixed, which is not optimal in all cases. Alternatively, as shown in FIG. 15B, a free-standing injection port 15c may be separate from the expander. The fill tube 15e can be cut to the appropriate length and then the free-standing injection port 15c may be attached to the fill tube 15e with a connector 15d. Sutures 15f may then be used to secure the connector 15d. This necessitates an extra step in surgery and the attachment has inherent problems of becoming loose and being bulky making removal more difficult.

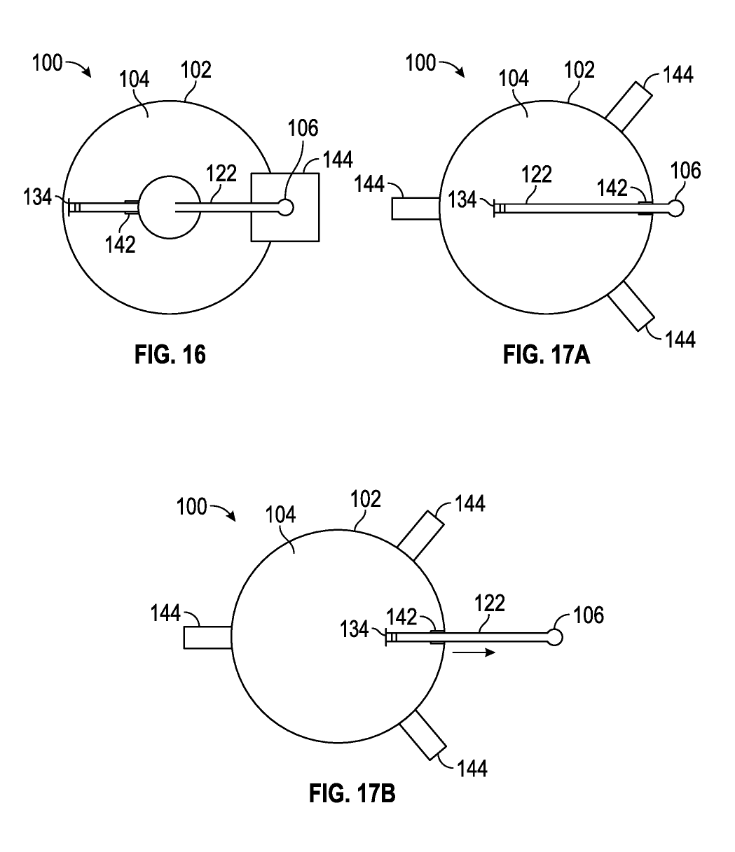

It should be clearly understood that the adjustable breast implant 100 may be used either as a temporary expander, which may later be removed and replaced with a silicone gel implant, or the adjustable breast implant 100 may be used permanently as described above. FIG. 16-17B show the adjustable breast implant 100 of the present invention. The adjustable breast implant 100 may have a retractable injection port 106 that is fixed to the fluid tube 122. The fluid tube 122 passes through the outer shell 102 and enters the inner lumen 104 through a valve that allows the length of the fluid tube 122 to be adjusted. A sealing cuff 142 allows the fluid tube 122 to elongate. A plug 134 may also be used to allow sealing should the injection port 106 need to be removed. If the injection port 106 is flush against the outer shell 102 it can function like an integral port expander. If it is necessary to place the injection port 106 at a remote site, the length of the fluid tube 122 may be extended (see FIG. 17B). If it is necessary to remove the injection port 106, e.g. prior to radiation or for MRI investigation, the injection port 106 can be removed by pulling on the fluid tube 122 until the plug 134 seals the adjustable breast implant 100. The adjustable breast implant 100 can now remain in position until it is replaced with a silicone implant at a second stage if the patient so desires. A suture tab 144 may also be placed proximate the injection port 106.

FIG. 17A-17B illustrates another advantage of the present invention, which is to place suture tabs 144 at the edges of the outer shell 102 for fixation. Although suture tabs are described in other expanders, what is advocated here is that the use of absorbable suture tabs 144. The absorbable suture tabs 144 may be constructed of polyglactin 910 (VICRYL®), polydioxanone (PDS®), poly-4-hydroxybutyrate (PHASIX®), or other similar absorbable meshes. The advantages of using an absorbable mesh for the suture tabs 144 include: no long-term thickened areas where the suture tab 144 is adherent; easier removal of the adjustable breast implant 100; no need to dissect the adherent suture tab 144 free; and less bleeding. The absorbable suture tabs 144 can also be used to secure the injection port 106 in position in place integral to the adjustable breast implant 100. A doughnut-shaped absorbable suture tab 144 can also be placed around the injection port 106 to facilitate palpability.

### Statement of Use

The most common technique of performing breast reconstruction following mastectomy is by placing a saline-filled tissue expander beneath the pectoral muscle, expanding the tissues by injecting saline into the expander and then changing the expander at a second procedure to a gel implant. There are several disadvantages inherent in the technique that the adjustable breast implant 100 overcomes. It has been found that an adjustable breast implant 100 provides the advantages of a breast implant combined with a tissue expander. The adjustable breast implant 100 provides advantages over conventional silicone implants or conventional saline implants because the adjustable breast implant 100 provides a silicone-like feel and is adjustable and can be permanently implanted with inclusion of an injection port 106 that is palpable in situ. Thus, the adjustable breast implant 100 may be adjusted years later after implantation.

The adjustable breast implant 100 that is either completely devoid of fluid or under-filled may be initially disposed in a patient to prevent any excess tension on a skin flap of an incision in which the adjustable breast implant 100 is disposed. Thereafter, the adjustable breast implant 100 may be filled with saline 112. When fully filled, the adjustable breast implant 100 does not ripple so the adjustable breast implant 100 may be permanently implanted and not replaced with another type of implant (e.g. gel implant).

When the adjustable breast implant 100 is fully filled, the inner bubble shells 128 expand, thereby baffling motion of saline 112 inside the outer shell 102 of the adjustable breast implant 100. As a result of baffling the motion of the saline 112 inside the outer shell 102, the adjustable breast implant 100 feels like a gel-filled implant even though gel can be absent in the adjustable breast implant 100. Accordingly, the adjustable breast implant 100 with this gel-like feel can remain in the patient without substituting the adjustable breast implant 100 with a silicone gel implant.

For a skin-sparing mastectomy, the adjustable breast implant 100 can be implanted above the pectoral muscle in the patient. Therefore, a small injection port 106 can be used to keep the adjustable breast implant 100 in position long term without the injection port 106 being bothersome to the patient.

The adjustable breast implant 100 may be used as a temporary expander, which may later be removed and replaced with a silicone gel implant, or the adjustable breast implant 100 may be used permanently as described above. If absorbable suture tabs 144 are used, the adjustable breast implant 100 may be removed after the absorbable suture tabs 144 have dissolved, if the patient so desires.

The foregoing description is illustrative of particular embodiments of the application, but is not meant to be limitation upon the practice thereof. While embodiments of the disclosure have been described in terms of various specific embodiments, those skilled in the art will recognize that the embodiments of the disclosure may be practiced with modifications within the spirit and scope of the claims.

What is claimed is:

1. An adjustable breast implant comprising:
an outer shell;
at least one inner lumen within the outer shell, wherein the at least one inner lumen is configured to receive a fluid to adjust a volume of the adjustable breast implant; and
an injection port disposed on the outer shell and positioned proximate an upper pole of the adjustable breast implant.

2. The adjustable breast implant of claim 1, wherein the adjustable breast implant comprises one inner lumen defined by the outer shell and wherein the fluid is received into the inner lumen via the injection port.

3. The adjustable implant of claim 1, wherein the injection port comprises:
a fill reservoir defined by the outer shell; and
a fluid tube connected to the fill reservoir;
wherein the fill reservoir is in fluid communication with the at least one inner lumen via the fluid tube.

4. The adjustable implant of claim 3, wherein the injection port further comprises an absorbable outer shell barrier.

5. The adjustable breast implant of claim 1 further comprising an inner shell disposed within the outer shell, wherein the outer shell defines a first inner lumen and wherein the inner shell defines a second inner lumen.

6. The adjustable breast implant of claim 5 wherein the first inner lumen contains a gel and wherein the second inner lumen receives the fluid to adjust a volume of the adjustable breast implant.

7. The adjustable breast implant of claim 5 wherein the inner shell separates the first inner lumen and the second inner lumen so that the inner shell blocks fluid communication between the first inner lumen and the second inner lumen.

8. The adjustable implant of claim 5 wherein the outer shell and the inner shell are attached to each other by the injection port so that the fluid may be injected into the second inner lumen via the injection port.

9. The adjustable breast implant of claim 1 further comprising:
a first inner lumen;
a plurality of bubble shells disposed within the first inner lumen, wherein the bubble shells are spaced apart from the outer shell and are positioned in a nested arrangement with respect to one another such that the bubble shells are spaced apart from each other by a plurality of second inner lumens, each second inner lumen being interposed between neighboring bubble shells;
wherein the bubble shells are size-adjustable based upon an amount of the fluid disposed therein; and
wherein the injection port provides fluid access to inflate and adjust a size of the first inner lumen and the second inner lumens.

10. The adjustable breast implant of claim 9 wherein each of the bubble shells has a three-dimensional sinusoidal shape and wherein the bubble shells baffle the fluid within the first inner lumen.

11. The adjustable breast implant of claim 1 further comprising:
one inner lumen; and
a plurality of bubble sheets disposed within the inner lumen;
wherein the bubble sheets baffle the fluid within the inner lumen.

12. The adjustable breast implant of claim 1 further comprising:
a plug positioned within the outer shell;
a hollow flow tube having one end coupled to the plug and having another end coupled to the injection port, wherein the fluid flows from the injection port through the hollow tube into the adjustable breast implant; and
an aperture disposed on the flow tube to provide a flow path of the fluid into the adjustable breast implant.

13. The adjustable breast implant of claim 12, wherein the injection port is removable from the plug via the flow tube whereby exerting a tensile force onto the injection port separates the injection port from the plug, and the plug seats against the outer shell in response to removal of the injection port due to the tensile force.

14. The adjustable breast implant of claim 13 further comprising a sealing patch disposed on the outer shell, wherein the sealing patch at least one of: seals the adjustable breast implant and prevents leakage of the fluid from the adjustable breast implant; sealingly engages the injection port prior to removal of the injection port; and sealingly engages the plug in response to removal of the injection port.

15. The adjustable breast implant of claim 1 further comprising a suture tab disposed proximate to the injection port at the upper pole of the adjustable breast implant.

16. An adjustable breast implant comprising:
an outer shell;
at least one inner lumen within the outer shell, wherein the at least one inner lumen is configured to receive a fluid to adjust a volume of the adjustable breast implant;
an injection port disposed on the outer shell and positioned proximate an upper pole of the adjustable breast implant, wherein the injection port comprises:
a fill reservoir defined by the outer shell; and
a fluid tube connected to the fill reservoir;
wherein the fill reservoir is in fluid communication with the at least one inner lumen via the fluid tube;
a suture tab disposed proximate to the injection port at the upper pole of the adjustable breast implant, wherein the suture tab is absorbable.

17. The adjustable breast implant of claim 16 further comprising an inner shell disposed within the outer shell, wherein the outer shell defines a first inner lumen that contains silicone gel, and wherein the inner shell defines a second inner lumen that contains saline.

18. The adjustable breast implant of claim 16 further comprising:
a first inner lumen;
a plurality of bubble shells disposed within the first inner lumen, wherein the bubble shells are spaced apart from the outer shell and are positioned in a nested arrangement with respect to one another such that the bubble shells are spaced apart from each other by a plurality of second inner lumens, each second inner lumen being interposed between neighboring bubble shells;
wherein each of the bubble shells have a three-dimensional sinusoidal shape, wherein the bubble shells baffle the fluid within the first inner lumen, and wherein the bubble shells are size-adjustable based upon an amount of the fluid disposed therein; and
wherein the injection port provides fluid access to inflate and adjust a size of the first inner lumen and the second inner lumens.

19. The adjustable breast implant of claim 16 further comprising:
a plug positioned within the outer shell;
a hollow flow tube having one end coupled to the plug and having another end coupled to the injection port, wherein the fluid flows from the injection port through the hollow tube into the adjustable breast implant; and
an aperture disposed on the flow tube to provide a flow path of the fluid into the adjustable breast implant;
wherein the injection port is removable from the plug via the flow tube whereby exerting a tensile force onto the injection port separates the injection port from the plug, and the plug seats against the outer shell in response to removal of the injection port due to the tensile force.

20. An adjustable breast implant comprising:
an outer shell;
at least one inner lumen within the outer shell, wherein the at least one inner lumen is configured to receive a fluid to adjust a volume of the adjustable breast implant;
an injection port disposed on the outer shell and positioned proximate an upper pole of the adjustable breast implant, wherein the injection port comprises:
a fill reservoir defined by the outer shell; and
a fluid tube connected to the fill reservoir;
an absorbable outer shell barrier;
wherein the fill reservoir is in fluid communication with the at least one inner lumen via the fluid tube;
an absorbable suture tab disposed proximate to the injection port at the upper pole of the adjustable breast implant;
a plug positioned within the outer shell;
a hollow flow tube having one end coupled to the plug and having another end coupled to the injection port, wherein the fluid flows from the injection port through the hollow tube into the adjustable breast implant; and
an aperture disposed on the flow tube to provide a flow path of the fluid into the adjustable breast implant;
wherein the injection port is removable from the plug via the flow tube whereby exerting a tensile force onto the injection port separates the injection port from the plug, and the plug seats against the outer shell in response to removal of the injection port due to the tensile force.
